(19) United States
(12) Patent Application Publication
(10) Pub. No.: US 2014/0315291 A1
(43) Pub. Date: Oct. 23, 2014

(54) Solar Conversion System And Methods

(71) Applicant: Superior Ecotech LLC, Boulder, CO (US)

(72) Inventors: Daniel Higgs, Boulder, CO (US); Philip Calabrese, Boulder, CO (US); Martin Gross, Ames, IA (US)

(73) Assignee: Superior Ecotech LLC, Boulder, CO (US)

(21) Appl. No.: 14/256,896

(22) Filed: Apr. 18, 2014

Related U.S. Application Data
(60) Provisional application No. 61/813,569, filed on Apr. 18, 2013.

(51) Int. Cl. C12M 1/00; C12M 1/02

(52) U.S. Cl. CPC C12M 21/02; C12M 27/00
USPC 435/292.1; 435/289.1

(57) ABSTRACT

Generally, culturing systems and methods for making and using culturing systems. Specifically, photobioreactor systems and methods for making and using photobioreactor systems to culture photosynthetic organisms, such as algae.

This United States Non-Provisional Patent Application claims the benefit of U.S. Provisional Patent Application No. 61/813,569, filed Apr. 18, 2013, hereby incorporated by reference herein.

### I. FIELD OF THE INVENTION

Generally, culturing systems and methods for making and using culturing systems. Specifically, photobioreactor systems and methods for making and using photobioreactor systems to culture photosynthetic organisms, such as algae.

### II. BACKGROUND OF THE INVENTION

Algae are a very large and diverse group of eukaryotic organisms, with over 40,000 identified species, that may use mineral nutrients and photon energy for growth and reproduction. By utilizing photon energy from sources such as sunlight or artificial light, algae may convert, through photosynthesis, water and carbon dioxide into high-value organic compounds (for example, pigments, proteins, carbohydrates, lipids, fatty acids, and secondary metabolites). Due to their extraordinarily efficient light and nutrient utilization, algae may exhibit a growth potential an order of magnitude greater than higher plants. As such, algae may be responsible for about one-third of the net photosynthetic activity worldwide.

Examples of algae utilization may include: use of algae as a source of biochemicals for the production of nutraceuticals, health foods, food additives, vitamins, pharmaceuticals, natural dyes, or the like; use of algae as an animal feed supplement; use of algae as a pesticide; use of algae as a soil conditioner or biofertilizer; use of algae for bioremediation; use of algae for biodegradation; use of algae as a renewable source for the production of a diesel fuel substitute (biodiesel) or other biofuels, such as ethanol, methane gas, hydrogen, or the like. With so many uses, it may be desirable to mass produce algae in a convenient, low-cost, high-yield manner.

A variety of conventional methods may exist for cultivating algae, including open systems or closed systems. Examples of conventional open systems may include lakes, ponds, raceways, or the like. These conventional open systems may have advantages, including relatively simple construction and maintenance. However, numerous disadvantages may be associated with conventional open systems, including lack of temperature control or light control, inadequate mixing, or the like, or combinations thereof. Conventional open systems may also be susceptible to culture contamination with airborne microorganisms, dust, or the like, or combinations thereof, which may result in culture failure. These and other significant drawbacks of conventional open systems for algae cultivation have prompted the development of closed systems, including conventional tubular-type photobioreactors, conventional flat plate-type photobioreactors, or the like.

Conventional tubular-type photobioreactors may be generally serpentine or helical in form, may be made of glass, plastic, or the like, and may include a gas exchange vessel, in which carbon dioxide and nutrients may be added and oxygen may be removed, connected to two ends of tubing. Recirculation of the culture between the gas exchange vessel and the tubing may be performed by a pump or an air-lift. As a result of their design, conventional tubular-type photobioreactors may increase biomass productivity. Nevertheless, conventional tubular-type photobioreactors may suffer from inherent problems, such as a substantial ‘dark zone or dark volume’, which may comprise about 10-15% of the total culture volume. As a result, tubular-type photobioreactors may only sustain a biomass yield of about 85-90% of the theoretical maximum. Additionally, high concentrations of molecular oxygen derived from photosynthesis may accumulate in the culture suspension within conventional tubular-type photobioreactors, which may inhibit photosynthesis and thus, decrease biomass productivity. Furthermore, mechanical pumps which may be employed by conventional tubular-type photobioreactors to facilitate culture mixing and circulation within the tubing may cause considerable algae cell damage, thereby limiting the number of algae species which may be cultivated within a conventional tubular-type bioreactor. Also, the high set-up and maintenance costs associated with conventional tubular-type photobioreactors may restrict their applications.

Another closed system for algae cultivation may include conventional flat plate-type photobioreactors, which may offer several advantages over conventional tubular-type photobioreactors, for example: absence of a “dark zone”, as conventional flat plate-type photobioreactors may be illuminated in their entirety, hence boosting photosynthesis and biomass productivity; aeration-facilitated culture mixing and turbulence, which may exert lesser hydrodynamic forces upon the algae cells relative to mechanical pumps; lower concentrations of molecular oxygen derived from photosynthesis resulting from lesser reactor heights; capacity to be oriented for maximal exposure to solar energy, which may enhance photosynthesis and biomass productivity; and, considerably less set-up and maintenance costs. However, there may be at least one major shortcoming of conventional flat plate-type photobioreactors, namely the challenge of scaling up these photobioreactors to a commercial level.

Thus, a need exists for a large-scale commercial production system that may provide cost-effective installation, operation, and maintenance relative to production yields. Additionally, to increase production yields, a system that increases photosynthesis may be desirable.

To increase the growth of photosynthetic organisms, light may be provided at an optimal intensity and an optimal frequency. When light conditions are not optimal, the growth of algae may be limited; for example, undesirable light intensity or light frequencies may cause a build-up of heat, may induce photorespiration, or may bleach the pigments needed for growth. These as well as other problems may be readily apparent in production systems that rely solely on direct solar light as a driver of photosynthesis, such as in lakes, ponds, or raceways, where algae cultures may be subject to light intensities and light frequencies which may be unusable by the culture. In addition, algae cultures within these production systems may be subject to diurnal or seasonal variability of light.

Thus, the ability to effectively control light may increase photosynthesis which may, in turn, increase production yields of algae and algae-associated products, making algae cultivation commercially viable. As commercial acceptance of algae cultivation may be dependent upon a variety of factors, including cost to manufacture, cost to operate, reliability, durability, scalability, or the like, or combinations thereof, culturing systems and methods for making and using culturing systems which may increase biomass production and decrease biomass production costs may be desirable.

### III. SUMMARY OF THE INVENTION

A broad object of a particular embodiment of the invention can be to provide a culturing system including a circuitous member, a biofilm adhesion surface coupled to the circuitous member, an axial element which supports the circuitous member in a circuitous travel path, a drive mechanism operable to advance the circuitous member in the circuitous travel path, and an optical filter disposed to filter light incident upon a biofilm adhering to the biofilm adhesion surface.

Another broad object of a particular embodiment of the invention can be to provide a method for producing a particular embodiment of the culturing system.

Another broad object of a particular embodiment of the invention can be to provide a method for using a particular embodiment of the culturing system.

Naturally, further objects of the invention are disclosed throughout other areas of the specification, drawings, and claims.

### IV. BRIEF DESCRIPTION OF THE DRAWINGS

FIG. 1 is an illustration of a method of using a particular embodiment of the inventive culturing system.

FIG. 2A is a side view of a component of a particular embodiment of the inventive culturing system, exaggerated for the purpose of illustration.

FIG. 2B is an exploded side view of the component of a particular embodiment of the inventive culturing system shown in FIG. 2A, exaggerated for the purpose of illustration.

FIG. 2C is a cross sectional view 2C-2C of a component of a particular embodiment of the inventive culturing system, exaggerated for the purpose of illustration.

FIG. 2D is an exploded cross sectional 2D-2D view of the component of a particular embodiment of the inventive culturing system shown in FIG. 2C, exaggerated for the purpose of illustration.

FIG. 3A is a side view of a component of a particular embodiment of the inventive culturing system.

FIG. 3B is an end view of the component of a particular embodiment of the inventive culturing system shown in FIG. 3A.

FIG. 3C is a side view of a component of a particular embodiment of the inventive culturing system.

FIG. 3D is an end view of the component of a particular embodiment of the inventive culturing system shown in FIG. 3C.

FIG. 3E is a side view of a component of a particular embodiment of the inventive culturing system.

FIG. 3F is an end view of the component of a particular embodiment of the inventive culturing system shown in FIG. 3E.

FIG. 4A is a perspective view of a component of a particular embodiment of the inventive culturing system.

FIG. 4B is a side view of a component of a particular embodiment of the inventive culturing system.

FIG. 4C is a side view of a component of a particular embodiment of the inventive culturing system.

FIG. 4D is a side view of a component of a particular embodiment of the inventive culturing system.

FIG. 4E is a side view of a component of a particular embodiment of the inventive culturing system.

FIG. 5A is a perspective view of a component of a particular embodiment of the inventive culturing system.

FIG. 5B is a side view of a component of a particular embodiment of the inventive culturing system.

FIG. 5C is an end view of a component of a particular embodiment of the inventive culturing system.

FIG. 5D is a top view of a component of a particular embodiment of the inventive culturing system.

FIG. 6 is a perspective view of a particular embodiment of the inventive culturing system.

FIG. 7 is a perspective view of a particular embodiment of the inventive culturing system.

FIG. 8 is a first side view of a particular embodiment of the inventive culturing system.

FIG. 9 is a second side view of a particular embodiment of the inventive culturing system.

FIG. 10 is a first end view of a particular embodiment of the inventive culturing system.

FIG. 11 is a second end view of a particular embodiment of the inventive culturing system.

FIG. 12 is a top view of a particular embodiment of the inventive culturing system.

FIG. 13 is a bottom view of a particular embodiment of the inventive culturing system.

FIG. 14A is a side view of a particular embodiment of the inventive culturing system.

FIG. 14B is a side view of a component the particular embodiment of the inventive culturing system shown in FIG. 14A, exaggerated for the purpose of illustration.

FIG. 15 is a side view of a particular embodiment of the inventive culturing system.

### V. DETAILED DESCRIPTION OF THE INVENTION

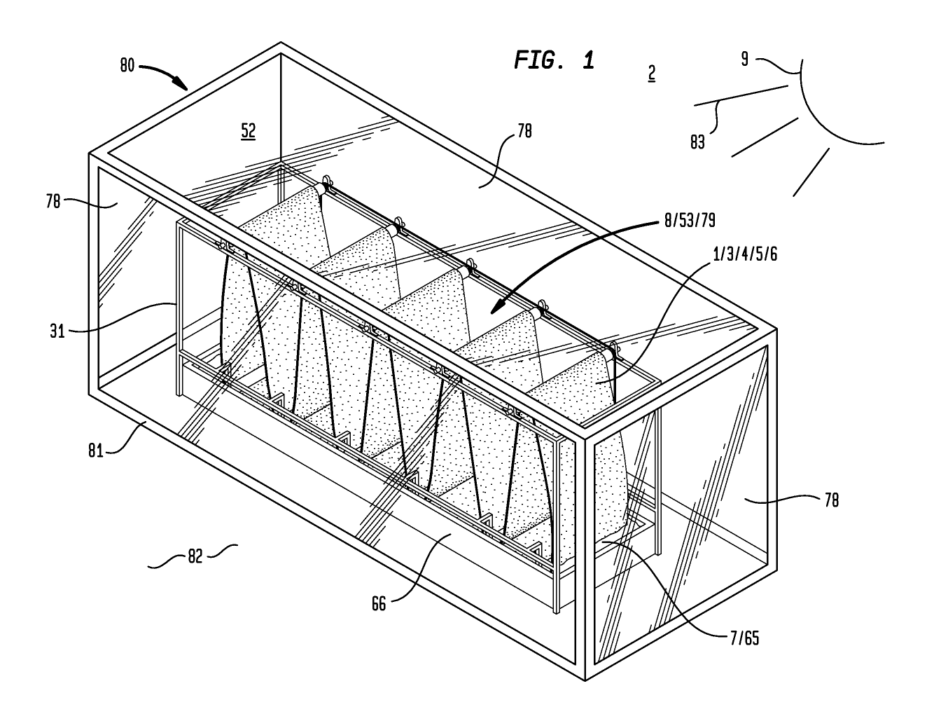

Now referring primarily to FIG. 1, which illustrates a method for culturing a biofilm (1), which can include providing a particular embodiment of the inventive culturing system (2), including a biofilm adhesion surface (3) coupled to a circuitous member (4). A biofilm (1) can be cultured by providing photosynthetic organisms (5), such as algae (6), to the inventive culturing system (2), wherein the photosynthetic organisms (5) can adhere to the biofilm adhesion surface (3) to form the biofilm (1); providing a biofilm culture medium (7) through which the biofilm (1) can advance in a circuitous travel path (8); and providing a light source (9) incident upon the biofilm (1) coupled to the circuitous member (4).

The term “photosynthetic particle” for the purposes of this invention means a natural organism, an engineered organism, a component of a natural organism, a component of an engineered organism, or the like, or combinations thereof, which can use light energy to synthesize organic compounds.

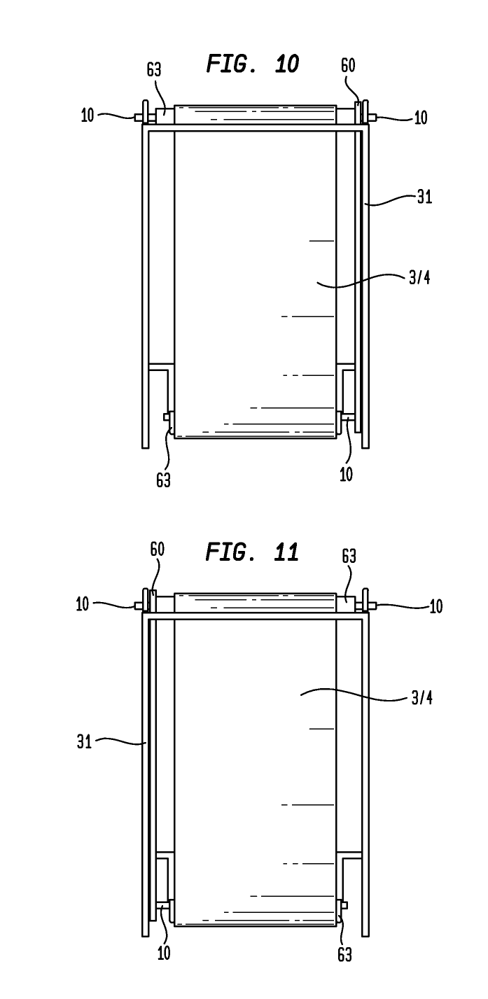

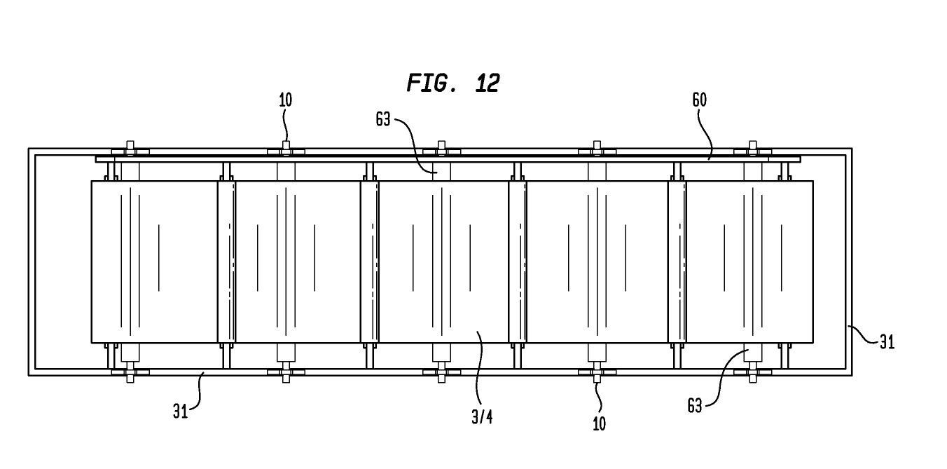

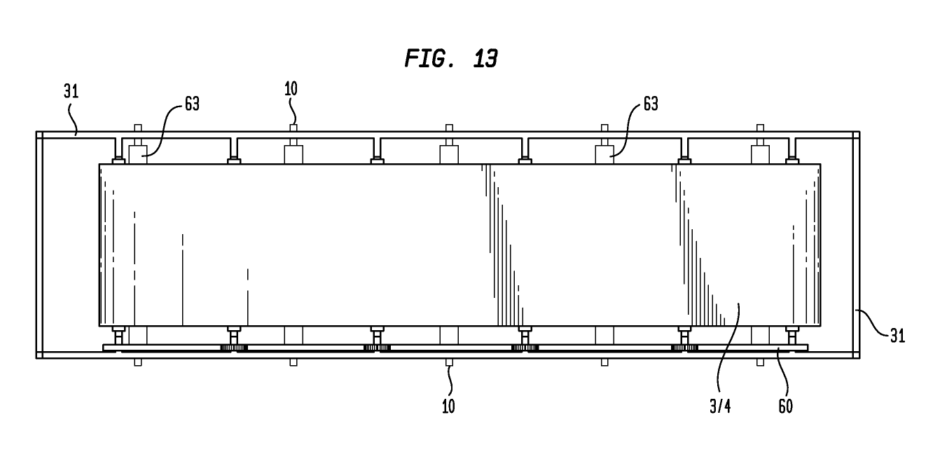

Now referring primarily to FIG. 6 through FIG. 15, the inventive culturing system (2) can include a circuitous member (4), a biofilm adhesion surface (3) coupled to the circuitous member (4), an axial element (10) which supports the circuitous member (4) in a circuitous travel path (8), and a drive mechanism (11) operable to advance the circuitous member (4) in the circuitous travel path (8).

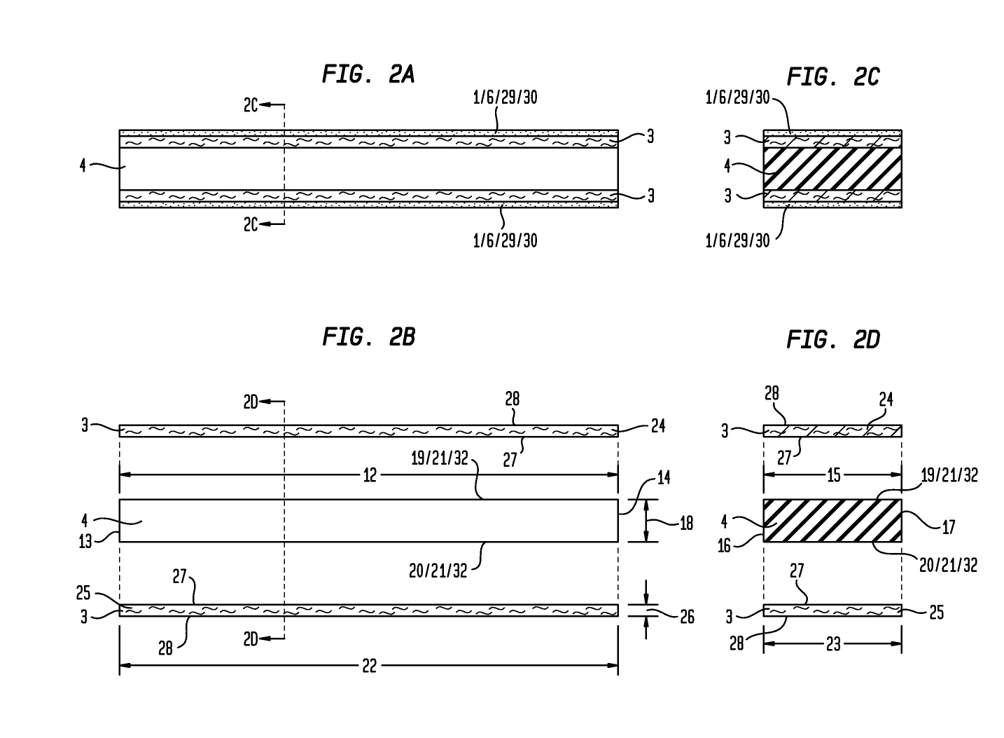

Now referring primarily to FIG. 2, the circuitous member (4) can include a circuitous member length (12) disposed between a circuitous member first end (13) joined to a circuitous member second end (14). Typically, the circuitous member length (12) can be in a range of between about 1 meter to about 1000 meters; however, embodiments can have a lesser or greater circuitous member length (12) depending upon the application.

Now referring primarily to FIG. 2, the circuitous member (4) can include a circuitous member width (15) disposed between a circuitous member first side (16) and a circuitous member second side (17). Typically, the circuitous member width (15) can be in a range of between about 0.1 meters to about 50 meters; however, embodiments can have a lesser or greater circuitous member width (15) depending upon the application.

As to particular embodiments, the circuitous member width (15) can be substantially uniform along the circuitous member length (12). As to other particular embodiments, the circuitous member width (15) can vary along the circuitous member length (12).

Now referring primarily to FIG. 2, the circuitous member (4) can include a circuitous member thickness (18) disposed between a circuitous member first surface (19) and a circuitous member second surface (20). Typically, the circuitous member thickness (18) can be in a range of between about 1 millimeter to about 50 millimeters; however, embodiments can have a lesser or greater circuitous member thickness (18) depending upon the application.

As to particular embodiments, the circuitous member thickness (18) can be substantially uniform over the circuitous member surface area (21) defined by circuitous member length (12) and the circuitous member width (15). As to other particular embodiments, the circuitous member thickness (18) can vary over the circuitous member surface area (21).

As an illustrative example, a circuitous member (4) can have a circuitous member length (12) of about 6 meters, a circuitous member width (15) of about 1.2 meters, and a substantially uniform circuitous member thickness (18) of about 12 millimeters.

Now referring primarily to FIG. 2, the inventive culturing system (2) can include a biofilm adhesion surface (3) coupled to the circuitous member (4). As to particular embodiments, the biofilm adhesion surface (3) can be coupled to the circuitous member first surface (19). As to other particular embodiments, the biofilm adhesion surface (3) can be coupled to the circuitous member second surface (20). As to other particular embodiments, the biofilm adhesion surface (3) can be coupled to the circuitous member first surface (19) and the circuitous member second surface (20). As to yet other particular embodiments, the biofilm adhesion surface (3) can be coupled to substantially the entirety or the entirety of the circuitous member external surface.

Now referring primarily to FIG. 2, as to particular embodiments, the biofilm adhesion surface (3) can have a biofilm adhesion surface length (22) and a biofilm adhesion surface width (23) of dimensions substantially similar to the corresponding dimensions of the circuitous member (4). As an illustrative example, a first biofilm adhesion surface (24) can be coupled to the circuitous member first surface (19) and a second biofilm adhesion surface (25) can be coupled to the circuitous member second surface (20), wherein the circuitous member length (12), the first biofilm adhesion surface length (22), and the second biofilm adhesion surface length (22) can be about 6 meters and the circuitous member width (15), the first biofilm adhesion surface width (23), and the second biofilm adhesion surface width (23) can be about 1.2 meters.

As to other particular embodiments, the biofilm adhesion surface length (22) can be dissimilar to the circuitous member length (12) or the biofilm adhesion surface width (23) can be dissimilar to the circuitous member width (15). As an illustrative example, the biofilm adhesion surface (3) can be coupled to a portion of the circuitous member (4), thereby coupling to less than the entirety of the circuitous member length (12) or less than the entirety of the circuitous member width (15).

Now referring primarily to FIG. 2, the biofilm adhesion surface (3) can have a biofilm adhesion surface thickness (26) disposed between a biofilm adhesion surface internal surface (27) and a biofilm adhesion surface external surface (28). Typically, the biofilm adhesion surface thickness (26) can be in a range of between about 1 millimeter to about 50 millimeters; however, embodiments can have a lesser or greater biofilm adhesion surface thickness (26) depending upon the application.

Now referring primarily to FIG. 2, which includes a circuitous member (4) disposed between a first biofilm adhesion surface (24) and a second biofilm adhesion surface (25), the first biofilm adhesion surface internal surface (27) can be coupled to the circuitous member first surface (19) and the second biofilm adhesion surface internal surface (27) can be coupled to the circuitous member second surface (20).

Now referring primarily to FIG. 1, FIG. 2A, and FIG. 2C, the inventive culturing system (2) can further include a biofilm (1) capable of adhering to the biofilm adhesion surface (3). As to particular embodiments, the biofilm (1) can comprise a plurality of photosynthetic particles (29). As to particular embodiments, the photosynthetic particles (29) can comprise a plurality of photosynthetic organisms (30), which can, but do not necessarily, contain chlorophyll, including as illustrative examples: a eukaryote containing chlorophyll such as a plant, a prokaryote containing chlorophyll such as cyanobacteria, or components thereof, or the like. As to particular embodiments, the photosynthetic organisms (30) can comprise algae (6), including unicellular algae or multicellular algae. As to particular embodiments, the algae (6) can be selected from the group including or consisting of: Axodine, Bacillariophyceae (for example Phaeodactylum tricornutum), Bryopsidophyceae, Chlorophyceae (for example Haematococcus pluvialis), Cyanophyceae (for example Spirulina), Dinophyceae (for example Crypthecodinium cohnii), Eustigmatophyceae (for example Nannochloropsis or Monodus subterraneus), Labyrinthulea (for example Schizochytrium or Thraustochytrium aggregatum or Ulkenia), Mesostigmatophyceae, Pelagophyceae, Phaeophyceae, Phaeothamniophyceae, Pleurastrophyceae, Prasinophyceae, Raphidophyceae, Synurophyceae, Trebouxiophyceae (for example Chlorella), Ulvophyceae, Xanthophyceae, or the like, or combinations thereof.

Now referring primarily to FIG. 2, the biofilm adhesion surface (3) can be comprised of any of a wide variety of materials capable of being adhered to by a biofilm (1). As to particular embodiments, the biofilm adhesion surface (3) can comprise a textured material, including woven fabric or film. The textured material can not only provide a substrate for the biofilm (1) to adhere to but can also increase the surface area to which the biofilm (1) can adhere to. As an illustrative example, the biofilm adhesion surface (3) can be produced from one or more of any of a wide variety of woven fabrics, including or consisting of: synthetic woven fabrics, natural woven fabrics (such as cotton), or the like, or combinations thereof. As an additional illustrative example, the biofilm adhesion surface (3) can be produced from one or more of any of a wide variety of films, including or consisting of: porous polymer film, plastic film, cellulose acetate film, polytetrafluoroethylene film, zeolite film, polyester film, nylon film, polypropylene film, acrylic film, polycarbonate film, titanium-coated film, polylignin film, or the like, or combinations thereof. As to particular embodiments, the film can be embedded with one or more of any of a wide variety of particles, including or consisting of: silicon dioxide nanoparticles, metal nanoparticles, organic nanoparticles, inorganic nanoparticles, or the like, or combinations thereof.

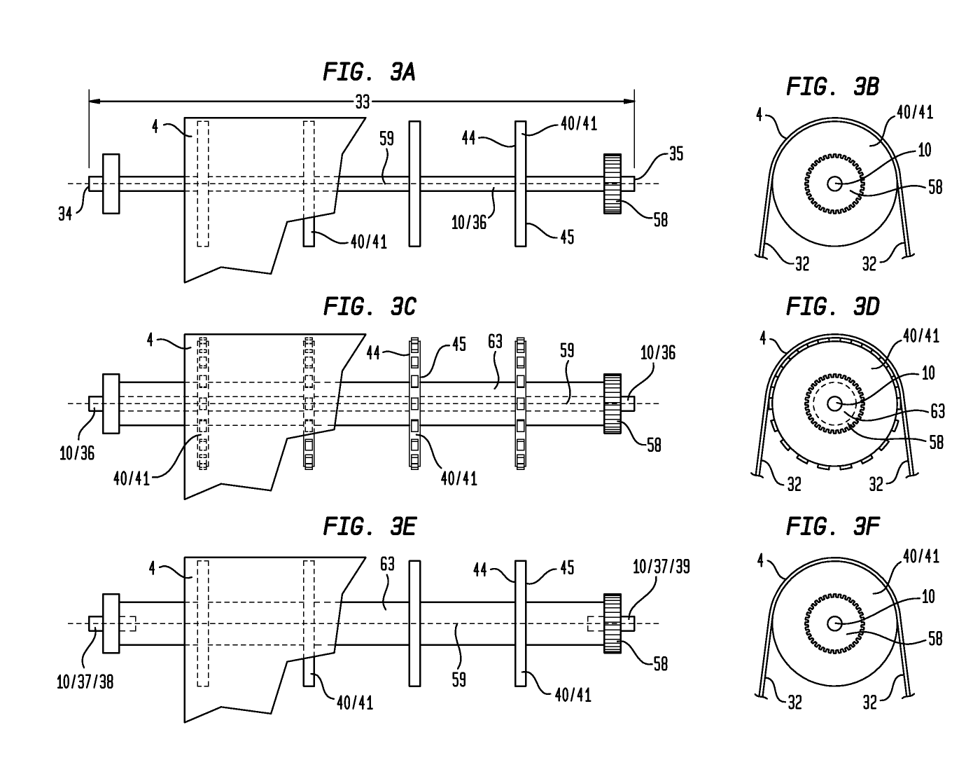

Now referring primarily to FIG. 3A through 3F, the inventive culturing system (2) can include an axial element (10) or a plurality of axial elements (10) which can support the circuitous member (4) in a circuitous travel path (8). The axial element (10) can comprise any of a wide variety of numerous configurations of varying dimensions, depending upon the application. As an illustrative example, the axial element (10) can comprise a rod or a tube configured as a cylinder, an ellipsoid, a prism, a cuboid, or the like, or combinations thereof. As to particular embodiments, a plurality of axial elements (10) can be coupled to one another, to a support frame (31) (as shown in the example of FIG. 1), or to the like, or combinations thereof. As to particular embodiments, the plurality of axial elements (10) coupled to one another or to a support frame (31) can be moveable; for example, one or more of the plurality of axial elements (10) or the support frame (31) can be mounted on a moveable assembly including, for example, wheels.

Now referring primarily to FIG. 3B, FIG. 3D, FIG. 3F, and FIG. 6 through FIG. 13, each axial element (10) can engage a corresponding portion of the circuitous member surface (32) to support the circuitous member (4). As to particular embodiments, the portion of the circuitous member surface (32) engaged by the axial element (10) can be the circuitous member first surface (19) or the circuitous member second surface (20). As to particular embodiments, the axial element (10) can be disposed across the circuitous member width (15) to support the circuitous member (4).

Now referring primarily to FIG. 3A, the axial element (10) can have an axial element length (33) disposed between an axial element first end (34) and an axial element second end (35). As to particular embodiments, an axial element (10) engaged to a corresponding portion of the circuitous member (4) across the circuitous member width (15) can be a continuous axial element (36) having an axial element length (33) which can be greater than the circuitous member width (15) (as shown in the example of FIG. 3A and FIG. 3C). As to other particular embodiments, an axial element (10) engaged to a corresponding portion of the circuitous member (4) across the circuitous member width (15) can be a discontinuous axial element (37) having a first discontinuous axial element portion (38) and a second discontinuous axial element portion (39), each having an axial element length (33) which can be lesser than the circuitous member width (15) (as shown in the example of FIG. 3E).

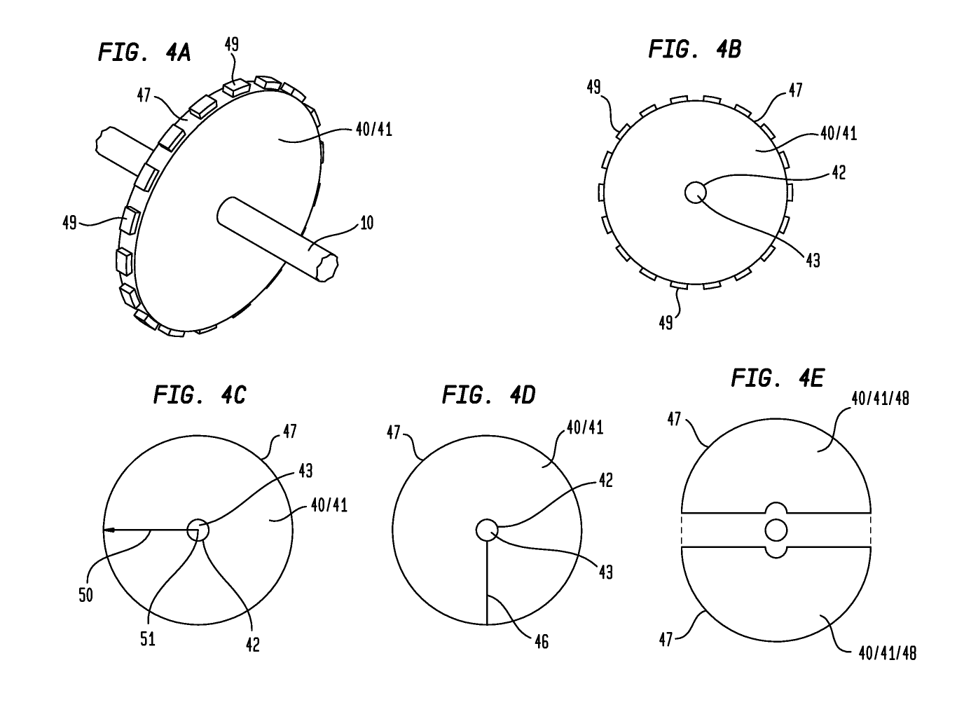

Now referring primarily to FIG. 3A through FIG. 4E, the inventive culturing system (2) can further include a spacer element (40) disposed between the axial element (10) and the corresponding portion of the circuitous member surface (32) which extends radially outward from the axial element (10). As to particular embodiments, a plurality of spacer elements (40) can be disposed in spaced apart relation along the axial element length (33).

Now referring primarily to FIG. 3A through FIG. 4E, the spacer element (40) can comprise any of a wide variety of numerous configurations of varying dimensions, depending upon the application. As to particular embodiments, the spacer element (40) can comprise an annular member (41) having an annular member aperture element (42) bounding an annular member aperture element opening (43), which communicates between an annular member first surface (44) and an annular member second surface (45). As to particular embodiments, the annular member (41) can include an annular member slit (46) communicating between the annular member aperture element opening (43) and the annular element circumferential periphery (47) (as shown in the example of FIG. 4D), wherein the axial element (10) can pass through the annular member slit (46) to the annular member aperture element opening (43) to dispose the annular member (41) about the axial element (10). As to other particular embodiments, the annular member (41) can include a plurality of annular member portions (48) (as shown in the example of FIG. 4E), wherein the plurality of annular element portions (48) can be joined together about the axial element (10) to dispose the annular member (41) about the axial element (10).

Now referring primarily to FIG. 4A, FIG. 4B, and FIG. 4C, the annular member (41) can have a textured annular element circumferential periphery (47); as an illustrative example, the annular member (41) can have a plurality of teeth (49) extending outward from the annular element circumferential periphery (47) (as shown in the example of FIG. 4A and FIG. 4B). As to other particular embodiments, the annular member (41) can have a relatively smooth annular element circumferential periphery (47) (as shown in the example of FIG. 4C).

Now referring primarily to FIG. 4C, the annular member (41) can have an annular member radius (50) disposed between the annular member center (51) and the annular element circumferential periphery (47). Typically, the annular member radius (50) can be in a range of between about 5 millimeters to about 100 millimeters; however, embodiments can have a lesser or greater annular member radius (50) depending upon the application. For example, a lesser annular member radius (50) can dispose the adjacent circuitous member surface (32) a lesser distance from the axial element (10). Conversely, a greater annular member radius (50) can dispose the adjacent circuitous member surface (32) a greater distance from the axial element (10).

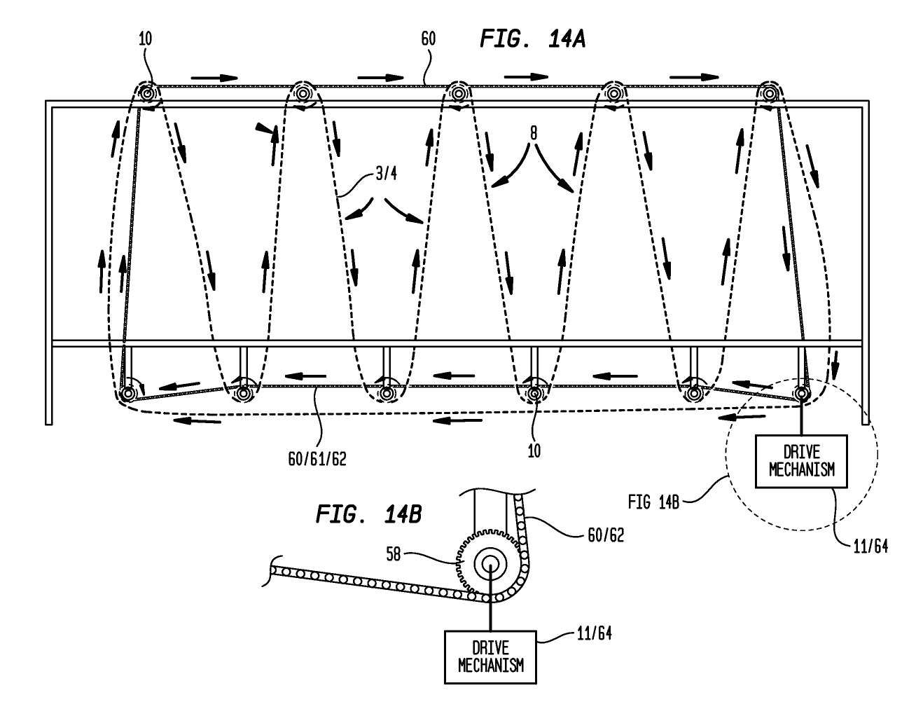

Now referring primarily to FIG. 1, FIG. 6 through FIG. 9, and FIG. 14A, the axial element (10) can support the circuitous member (4) in a circuitous travel path (8) (as shown by the arrows about the circuitous member (4) in FIG. 14A). As to particular embodiments, the circuitous travel path (8) can be defined by disposing one or more axial elements (10) in fixed spatial relation which supports the circuitous member (4) about the one or more axial elements (10) in a corresponding circuitous travel path (8) through which the circuitous member (4) can continuously advance. The circuitous travel path (8) can be defined by any of a wide variety of numerous configurations of varying dimensions by altering the fixed relation of the one or more axial elements (10), depending upon the application. As an illustrative example, the circuitous travel path (8) can be configured as a circle, an oval, an ellipse, a triangle, a square, a rectangle, a trapezoid, a polygon, or the like, or combinations thereof.

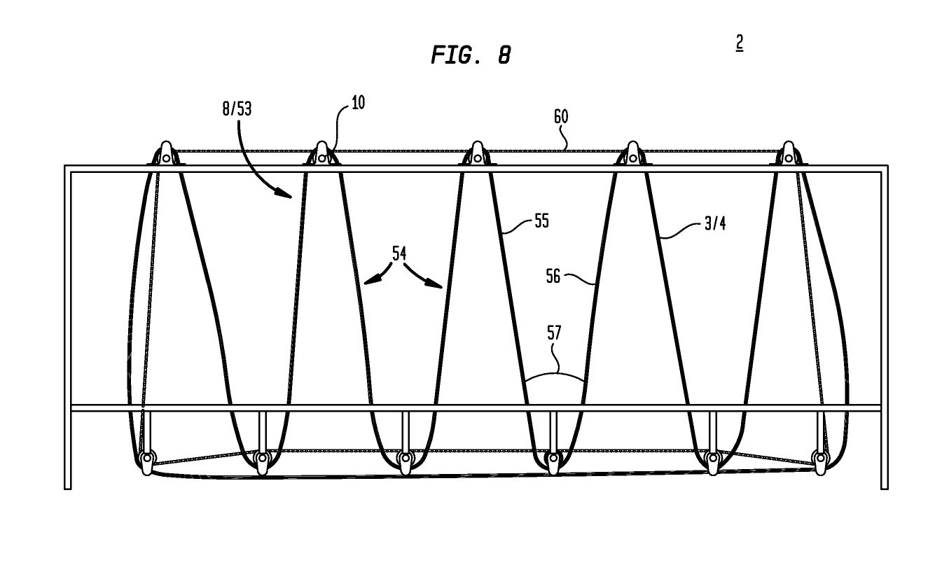

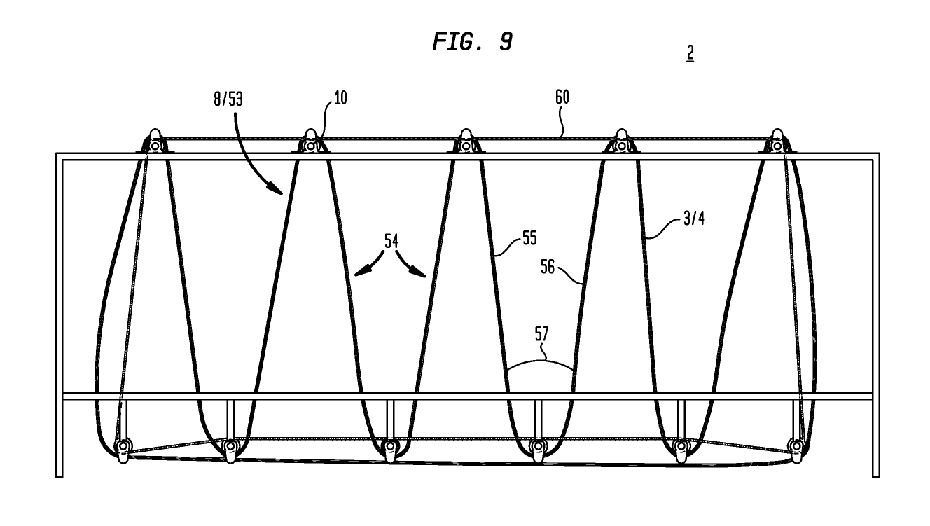

Now referring primarily to FIG. 7 through FIG. 9, the circuitous member (4) can be disposed about a plurality of axial elements (10) having a fixed spatial relation which can increase a circuitous member surface area (21) of the circuitous member (4) in fixed volume of space (52) or per unit volume of space (52). As to particular embodiments, the circuitous member (4) can have a folded configuration (53) which repeatedly disposes the circuitous member first surface (19) in opposed adjacent relation which increases the circuitous member surface area (21) in a fixed volume of space (52) or per unit volume of space (52) as compared to a circuitous member (4) having a planar configuration within the same fixed volume of space (52). As to particular embodiments, the circuitous member (4) can be disposed in a plurality of accordion folds (54). As to particular embodiments, a pair of adjacent accordion fold surfaces (55)(56) can be in substantially parallel relation to one another. As to other particular embodiments, a pair of adjacent accordion fold surfaces (55)(56) can be in angled relation to one another (as shown in the example of FIG. 8 and FIG. 9). The angle (57) between the pair of adjacent accordion fold surfaces (55)(56) can be in a range of between about 1 degree to about 179 degrees, depending upon the application. As to particular embodiments, a plurality of accordion folds (54) having lesser angles (57) between the pairs of adjacent accordion fold surfaces (55)(56) can increase the circuitous member surface area (21) in fixed volume of space (52) or per unit volume of space (52) relative to a plurality of accordion folds (54) having greater angles (57) between the pairs of adjacent accordion fold surfaces (55)(56). As to other particular embodiments, a plurality of accordion folds (54) having greater angles (57) between the pairs of adjacent accordion fold surfaces (55)(56) can increase exposure of the biofilm (1) to light.

Now referring primarily to FIG. 3A through FIG. 3F, the inventive culturing system (2) can further include a driven member (58) coupled to the axial element (10). As to particular embodiments, the drive mechanism (11) can drive the driven member (58), rotating the driven member (58) about a rotation axis (59) to advance the circuitous member (4) in the circuitous travel path (8). As to particular embodiment, one driven member (58) can be coupled to the axial element (10) proximate one of the axial element ends (34)(35). As to other particular embodiments, two driven members (58) can be coupled to the axial element (10), one driven member (58) proximate each of the two axial element ends (34)(35).

Now referring primarily to FIGS. 14A and 14B, a plurality of driven members (58) can be coupled together by a circuitous drive element (60). As to particular embodiments, a drive mechanism (11) can drive a first of the plurality of driven members (58) which in turn can drive the remaining plurality of driven members (58) coupled to the circuitous drive element (60) (as shown by the arrows about the circuitous drive element (60) in FIG. 14A).

Now referring primarily to FIG. 14A and FIG. 14B, the circuitous drive element (60) can comprise any of a wide variety of numerous configurations of varying dimensions, depending upon the application. As an illustrative example, a circuitous drive element (60) can be a continuous belt (61), which can be coupled to a plurality of driven members (58) configured in a pulley system. As another illustrative example, the circuitous drive element (60) can be a roller chain (62), which can be coupled to a plurality of driven members (58), each configured as a toothed sprocket (63).

Now referring primarily to FIG. 3A, the driven member (58) can be fixedly coupled to the axial element (10) supporting the circuitous member (4). As such, rotation of the driven member (58) can result in rotation of the axial element (10) about the rotation axis (59), thereby advancing the circuitous member (4) in the circuitous travel path (8).

Now referring primarily to FIG. 3C and FIG. 3E, the driven member (58) can be rotatably coupled to the axial element (10) and fixedly coupled to a rotatable element (63) disposed between the axial element (10) and the circuitous member (4), wherein the rotatable element (63) can extend radially outward from the axial element (10). Rotation of the driven member (58) can result in rotation of the rotatable element (63) about the rotation axis (59), thereby advancing the circuitous member (4) in the circuitous travel path (8).

Now referring primarily to FIG. 14A and FIG. 14B, the inventive culturing system (2) can include a drive mechanism (11) operable to advance the circuitous member (4) in the circuitous travel path (8). As to particular embodiments, the drive mechanism (11) can be operatively coupled to the driven member (58), wherein rotation of the driven member (58) coupled to the circuitous member (4) advances the circuitous member (4) in the circuitous travel path (8). As to particular embodiments, the drive mechanism (11) can comprise any of a wide variety of mechanisms suitable for driving the driven member (58). As an illustrative example, the drive mechanism (11) can comprise a machine which converts energy into motion, such as a motor (64). As to particular embodiments, the motor (64) can be an electric motor, which can convert electrical energy into mechanical energy. The electrical energy can be provided by any of a wide variety of sources, including a direct current source, an alternating current source, solar energy, or the like, or combinations thereof.

Now referring primarily to FIG. 1 and FIG. 6, the inventive culturing system (2) can further include a biofilm culture medium (7) through which the circuitous member (4) can advance in the circuitous travel path (8). As illustrative examples, the biofilm culture medium (7) can include a conventional cell culture medium, water, saline water, groundwater, contaminated groundwater, agriculture runoff water, underground saline water, industrial wastewater, domestic wastewater, animal wastewater, exhaust gas, or the like, or combinations thereof. As to particular embodiments, the biofilm culture medium (7) can comprise an algae culture medium (65). Additionally, the biofilm culture medium (7) can be supplemented with various particular nutrients, which can be added in a dry powder or liquid form, including urea (CH₄N₂O), sodium nitrate (NaNO3), magnesium sulfate (MgSO₄), sodium chloride (NaCl), monopotassium phosphate (KH₂PO₄), dipotassium phosphate (K₂HPO₄), calcium chloride (CaCl₂), zinc sulfate (ZnSO₄), manganese chloride (MnCl₂), molybdenum trioxide (MoO₃), copper sulfate (CuSO₄), cobalt nitrate (CoNO₃), boric acid (H₃BO₃), ethylenediaminetetraacetic acid (C₁₀H₁₆N₂O₈), potassium hydroxide (KOH), iron sulfate (FeSO₄), sulfuric acid (H₂SO₄), or the like, or combinations thereof.

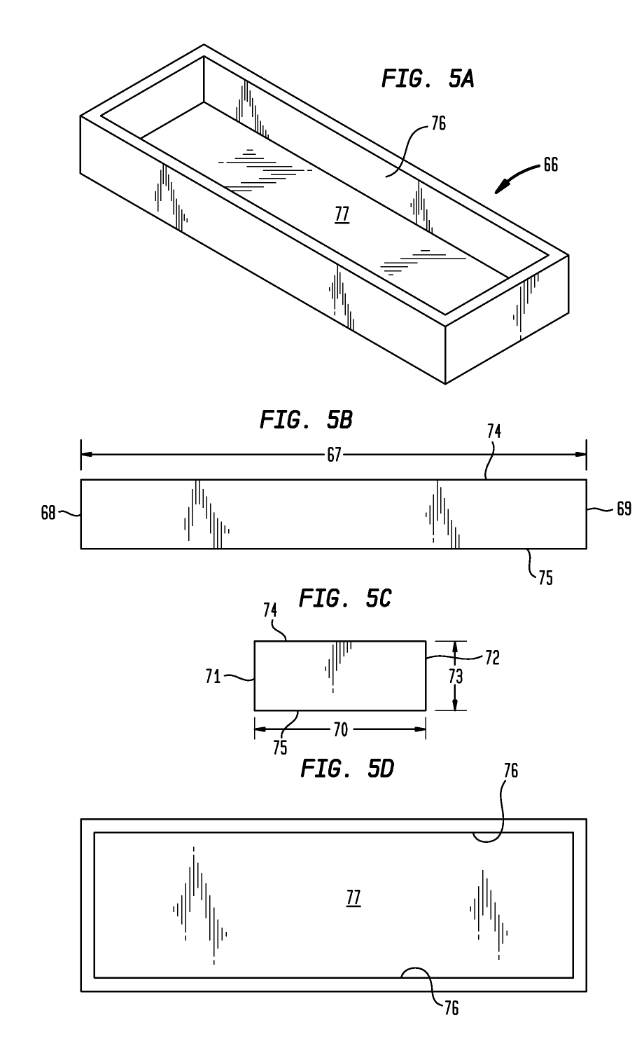

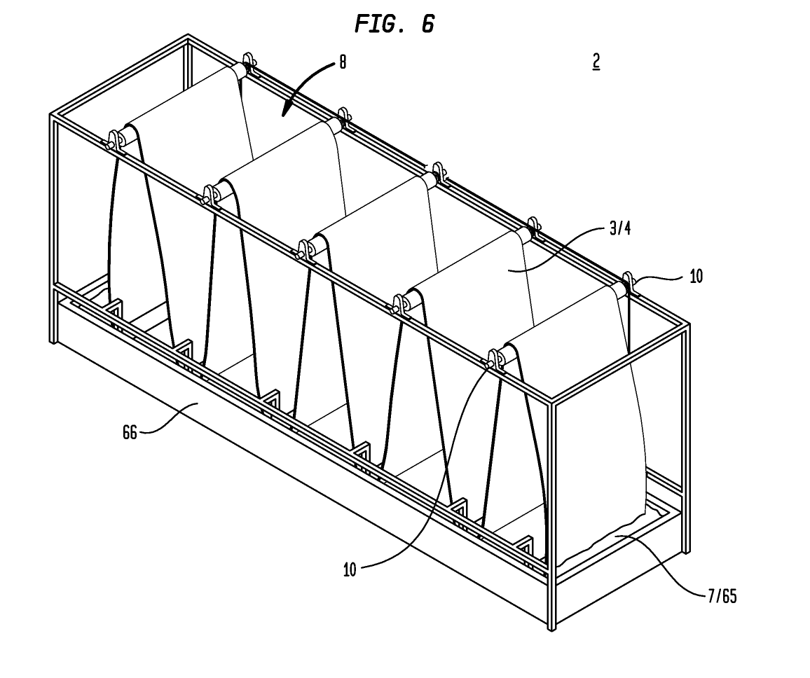

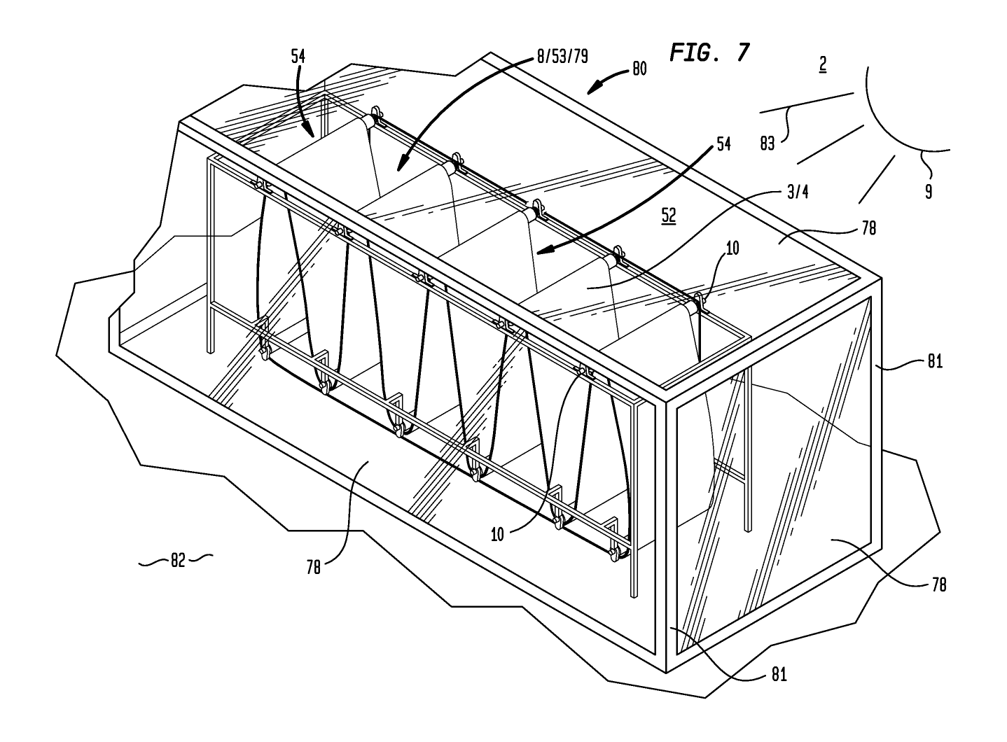

As to particular embodiments, the biofilm culture medium (7) can be contained within a lake, a pond, a raceway, or the like. Now referring primarily to FIG. 5A through FIG. 5D, the inventive culturing system (2) can further include a receptacle (66) containing the biofilm culture medium (7) through which the circuitous member (4) advances in a portion of the circuitous travel path (8). The receptacle (66) can have a receptacle length (67) disposed between a receptacle first end (68) and a receptacle second end (69). Typically, the receptacle length (67) can be in a range of between about 2 meters to about 50 meters; however, embodiments can have a lesser or greater receptacle length (67) depending upon the application. The receptacle (66) can also have a receptacle width (70) disposed between a receptacle first side (71) and a receptacle second side (72). Typically, the receptacle width (70) can be in a range of between about 0.5 meters to about 25 meters; however, embodiments can have a lesser or greater receptacle width (70) depending upon the application. The receptacle (66) can also have a receptacle height (73) disposed between a receptacle top portion (74) and a receptacle bottom portion (75). Typically, the receptacle height (73) can be in a range of between about 0.1 meters to about 5 meters; however, embodiments can have a lesser or greater receptacle height (73) depending upon the application.

Now referring primarily to FIG. 5A through FIG. 5D, the receptacle (66) can comprise any of a wide variety of numerous configurations of varying dimensions, depending upon the application. As an illustrative example, a substantially cuboidal receptacle (66) can have a receptacle length (67) of about 6 meters, a receptacle width (70) of about 1.5 meters, and a receptacle height (73) of about 0.5 meters.

Now referring primarily to FIG. 5A and FIG. 5D, the receptacle (66) can have a receptacle internal surface (76) which can define a receptacle internal space (77). As an illustrative example, a substantially cuboidal receptacle (66) can have a receptacle internal surface (76) which can define a substantially cuboidal receptacle internal space (77).

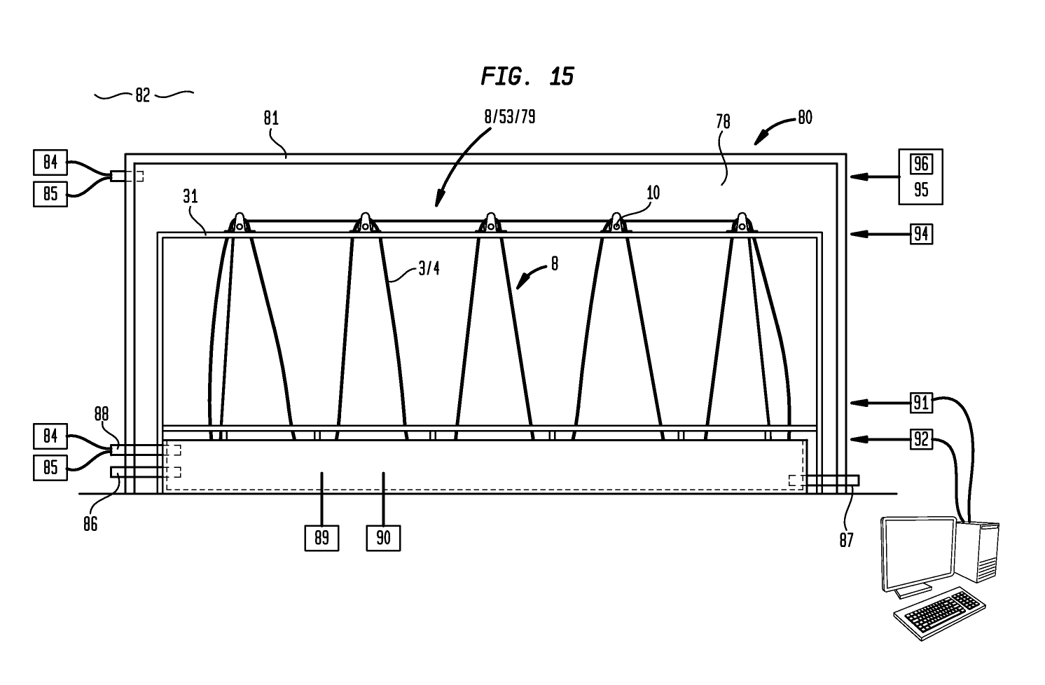

Now referring primarily to FIG. 1, FIG. 7, and FIG. 15, the inventive culturing system (2) can further include an optical filter (78) disposed to filter light incident upon the biofilm (1) coupled to the circuitous member (4). As to particular embodiments, the optical filter (78) can be disposed about the circuitous member (4). For example, an optical filter (78) can be disposed about an assembly (79) including a circuitous member (4) in the form of a plurality of accordion folds (54). As to particular embodiments, the optical filter (78) can be disposed in whole or in part about the assembly (79). As to particular embodiments, the optical filter (78) can be disposed in substantially parallel relation, substantially perpendicular relation, or in angled relation to the assembly (79). As to particular embodiments, the optical filter (78) can be substantially planar, arcuate, undulating, or the like, or combinations thereof.

As to particular embodiments, the optical filter (78) can include a plurality of optical filters (78) disposed about a corresponding plurality of portions of the assembly (79). The plurality of optical filters (78) can be substantially similar to one another or dissimilar from one another. As shown in the example of FIG. 1, FIG. 7, and FIG. 15, the optical filters (78) be located to dispose corresponding edges adjacent one another to form an enclosure (80) about the assembly (79). As to particular embodiments, a portion or the entirety of one or more of the plurality of optical filters (78) can be removable to provide ingress or egress from the enclosure (80).

Now referring primarily to FIG. 1, FIG. 7, and FIG. 15, the optical filter (78) can further include an optical filter framework (81) to which the plurality of optical filters (78) can couple in fixed relation to form the enclosure (80) or other configurations of the optical filter (78) about the assembly (79). Generally, the optical filter framework (81) can provide rigid support as well as a surface area to which the plurality of optical filters (78) can be coupled. As to particular embodiments, the enclosure (80) can be configured to regulate the transfer of one or more of solids, liquids, gases, heat, light, or the like, or combinations thereof, between the enclosure (80) or other configurations of optical filter and the ambient environment (82). As to particular embodiments, the enclosure (80) can be to a greater or lesser extent sealed from the ambient environment (82), and as to particular embodiments, hermetically sealed.

Now referring primarily to FIG. 1, FIG. 7, and FIG. 15, the optical filter (78) can transmit light corresponding to the entire or a portion of the ambient wavelengths or can transmit a predetermined wavelength. As to particular embodiments, the optical filter (78) can filter ambient light (83) to transmit light incident on the biofilm (1) coupled to the circuitous member (4) having one or more predetermined wavelengths which can modulate one or more parameters selected from the group including or consisting of: production of the biofilm (1), rate of growth of the biofilm (1), biomass of the biofilm (1), photosynthesis of the biofilm (1), production of a biochemical component by the biofilm (1), carbon dioxide consumption of the biofilm (1), or the like. As to particular embodiments, the biochemical component can be selected from the group including or consisting of: ethanol, lipids, polyunsaturated fatty acids, omega-3 fatty acids, carbohydrates, proteins, nucleic acids, pigments, antioxidants, carotenoids, beta carotene, lutein, astaxanthin, or the like.

As to particular embodiments, the optical filter (78) can transmit light having a wavelength in a range of between about 100 nanometers to about 2500 nanometers; however, embodiments of the optical filter (78) can transmit light having a lesser or greater wavelength or a predetermined wavelength depending upon the application. As to particular embodiments, one or more wavelengths can be selected from the group including or consisting of: between about 100 nanometers to about 150 nanometers, between about 125 nanometers to about 175 nanometers, between about 150 nanometers to about 200 nanometers, between about 175 nanometers to about 225 nanometers, between about 200 nanometers to about 250 nanometers, between about 225 nanometers to about 275 nanometers, between about 250 nanometers to about 300 nanometers, between about 275 nanometers to about 325 nanometers, between about 300 nanometers to about 350 nanometers, between about 325 nanometers to about 375 nanometers, between about 350 nanometers to about 400 nanometers, between about 375 nanometers to about 425 nanometers, between about 400 nanometers to about 450 nanometers, between about 425 nanometers to about 475 nanometers, between about 450 nanometers to about 500 nanometers, between about 475 nanometers to about 525 nanometers, between about 500 nanometers to about 550 nanometers, between about 525 nanometers to about 575 nanometers, between about 550 nanometers to about 600 nanometers, between about 575 nanometers to about 625 nanometers, between about 600 nanometers to about 650 nanometers, between about 625 nanometers to about 675 nanometers, between about 650 nanometers to about 700 nanometers, between about 675 nanometers to about 725 nanometers, between about 700 nanometers to about 750 nanometers, between about 725 nanometers to about 775 nanometers, between about 750 nanometers to about 800 nanometers, between about 775 nanometers to about 825 nanometers, between about 800 nanometers to about 850 nanometers, between about 825 nanometers to about 875 nanometers, between about 850 nanometers to about 900 nanometers, between about 875 nanometers to about 925 nanometers, between about 900 nanometers to about 950 nanometers, between about 925 nanometers to about 975 nanometers, between about 950 nanometers to about 1000 nanometers, between about 975 nanometers to about 1025 nanometers, between about 1000 nanometers to about 1500 nanometers, between about 1250 nanometers to about 1750 nanometers, between about 1500 nanometers to about 2000 nanometers, between about 1750 nanometers to about 2250 nanometers, and between about 2000 nanometers to about 2500 nanometers, or combinations thereof.

As to particular embodiments, the optical filter (78) can transmit light which the biofilm (1) can use to grow or proliferate and reduce transmission of light which may be harmful to the growth or proliferation of the biofilm (1). As an illustrative example, the optical filter (78) can reduce transmission of ultraviolet light in particular embodiments of the inventive culturing system (2) which include a biofilm (1) that can be negatively affected by ultraviolet light. As an additional illustrative example, the optical filter (78) can reduce transmission of infrared light in particular embodiments of the inventive culturing system (2) which include a biofilm (1) that can be negatively affected by infrared light.

As to particular embodiments, the optical filter (78) can include a nanolaminate comprising one or more layers of filter material. As to particular embodiments, the nanolaminate can include one or more compounds selected from the group including or consisting of: oxides, nitrides, carbides, metals, sulfides, fluorides, biomaterials, polymers, or the like. As to particular embodiments, the nanolaminate can be coupled to a base material, such as glass or plastic, to produce the optical filter (78). As to particular embodiments, the nanolaminate can be coupled to the base material by one or more methods selected from the group including or consisting of: chemical vapor deposition, hot-wire chemical vapor deposition, atomic layer deposition, molecular layer deposition, thermal evaporation, sputter deposition, cathodic arc deposition, electron beam physical vapor deposition, pulsed laser deposition, or the like.

Now referring primarily to FIG. 7 and FIG. 15, the inventive culturing system (2) can further include a light source (9) which emits an amount of light incident on the biofilm (1) coupled to the circuitous member (4). As to particular embodiments, the light source (9) can include ambient light (83), for example, natural light, sunlight, artificial light, or combinations thereof. As to particular embodiments, one or more optical fiber bundles can be configured to transmit light from the light source (9) to the biofilm (1).

As to particular embodiments, the light source (9) can be disposed at locations about the assembly (79) to direct an amount of light incident on the biofilm (1) coupled to the circuitous member (4). Artificial light can be desirable during periods of limited availability of natural light, such as during the night, during seasons with fewer hours of natural light, or during cloudy weather patterns. Artificial light can be produced by various conventional light sources (9) including incandescent lights, fluorescent lights, mercury vapor lights, light emitting diodes, or the like, or combinations thereof. As to particular embodiments, the light source (9) can be powered by electrical energy, solar energy, or the like, or combinations thereof. As to particular embodiments, a reflective surface, for example a mirror, can direct the amount of light incident on the biofilm (1) coupled to the circuitous member (3).

Now referring primarily to FIG. 15, the inventive culturing system (1) can further include a source of carbon dioxide (84), which can regulate the partial pressures of carbon dioxide (84) about the assembly (79). The source of carbon dioxide (84) can be, as illustrative examples, a container of pressurized carbon dioxide gas, a solution or suspension including carbon dioxide gas, yeast fermentation, exhaust gas, or the like, or combinations thereof, from which carbon dioxide (84) can be produced to regulate the partial pressures of carbon dioxide (84) about the assembly (79), within the enclosure (80), within the biofilm culture medium (7), or combinations thereof.

Now referring primarily to FIG. 15, the inventive culturing system (1) can further include a source of heat (85), which can be introduced into the volume of space (52) enclosed by the enclosure (80), the biofilm culture medium (7), or a combination thereof, which can be controlled to maintain the temperature within a desired range. As to particular embodiments, the source of heat (85) can include a heating element, such as a resistive heating element, to generate heat (85). As to other particular embodiments, the heat (85) can derive from exhaust gas.

Now referring primarily to FIG. 15, the inventive culturing system (2) can further include a liquid inlet port (86) or a liquid outlet port (87) in fluid communication with the receptacle (66), through which liquid can be added to the receptacle (66) or removed from the receptacle (66), respectively. By way of example, the liquid inlet port (86) can be used to introduce water, biofilm culture medium (7), algae (6), nutrient solutions or suspensions, or the like, or combinations thereof, into the receptacle (7) and the liquid outlet port (87) can be used to remove wastewater from the receptacle (66).

Now referring primarily to FIG. 15, the inventive culturing system (2) can further include a gas inlet port (88) in fluid communication with the volume of space (52) enclosed by the enclosure (80), the receptacle (66), or a combination thereof, which can be used to introduce gas into the volume of space (52) enclosed by the enclosure (80), the receptacle (66), or a combination thereof. By way of example, the gas can include carbon dioxide (84), carbon monoxide, oxygen, nitrogen, nitrogen-containing compounds (NOₓ), sulfur containing compounds (SOₓ), or the like, or combinations thereof.

Now referring primarily to FIG. 15, the inventive culturing system (2) can further include a fluid circulation system (89) coupled to the receptacle (66) and configured to force a continuous flow of biofilm culture medium (7) through the receptacle (66). The fluid circulation system (89) can force the continuous flow of biofilm culture medium (7) through the receptacle (66) by using any of a wide variety of pumping mechanisms including or consisting of: a positive displacement pumping system (for example, gear, rotary gear, rotary lobe, diaphragm, piston, screw, peristaltic, or the like), a rotodynamic pumping system (for example, centrifugal, radial flow, axial flow, mixed flow, injector, ejector, eductor-jet, or the like), a buoyancy-driven pumping system (for example, single-phase, multi-phase, or the like), a gravity-driven pumping system (for example, a sloped container, or the like), or a combination thereof.

Now referring primarily to FIG. 15, the inventive culturing system (2) can further include an aeration system (90) in fluid communication with the receptacle (66). The aeration system (90) can include any of a wide variety of systems suitable for introducing a gas supply into the receptacle (66), which can include tubing made of flexible or rigid plastic (for example, silicon or polyvinyl chloride), or metal (for example, stainless steel). As an illustrative example, the aeration system (90) can include compressed air passing though tubing disposed along the receptacle internal surface (76). As to particular embodiments, the aeration system (90) can provide carbon dioxide (84) for use as a carbon source for photosynthesis. Additionally, the aeration system (90) can provide gas bubbles, such as oxygen or carbon dioxide (84) to the biofilm culture medium (7), to effect culture mixing.

Now referring primarily to FIG. 15, the inventive culturing system (2) can further include sensors (91) and modulators (92) for controlling parameters of the inventive culturing system (2), which can include temperature, pH, wavelength of light, nutrient concentration, nitrate concentration, phosphate concentration, oxygen concentration, carbon dioxide concentration, biofilm (1) density, or the like, or combinations thereof. It can be appreciated that there are many sensors (91) and modulators (92) known to those of ordinary skill in the art which can be used to control one or more of the aforementioned parameters, any of which can be implemented using an automatic control system or methodology. For example, a computer-based control system (93) can be integrated into the inventive culturing system (2) to control one or more of the aforementioned parameters. As to particular embodiments, a sensor (91) or a modulator (92) can be in wired or wireless communication with the control system (93). As an illustrative example, an optical density sensor (91) can be wirelessly integrated into the inventive culturing system (2) for online monitoring of biofilm (1) density, which can be used to control harvesting of the biofilm (1).

Now referring primarily to FIG. 15, the inventive culturing system (2) can further include an inclination mechanism (94) to effect inclination of the inventive culturing system (2). As a direct relationship between solar energy and biofilm (1) productivity can generally be observed, wherein the greater the amount of solar energy transmitted into the inventive culturing system (2) by varying the inventive culturing system (2) tilt angle according to time of day or season, the greater the productivity of the biofilm (1). Thus, the inventive culturing system (2) tilt angle can exert a significant effect on the biofilm (1) density and accordingly, on the biofilm (1) productivity.

As to particular embodiments, the inventive culturing system (2) can further include a harvesting assembly (95), which can be used to harvest the biofilm (1). As to particular embodiments, the harvesting assembly (95) can include a shearing element (96), which can be manually or automatically operated. As an illustrative example, an optical density sensor (91) integrated into the inventive culturing system (2) can sense a biofilm (1) density associated with a biofilm (1) thickness of about 10 millimeters, wherein the shearing element (96) can be automatically activated to engage the biofilm (1) and harvest a portion of the biofilm (1) by shearing the upper 5 millimeters of the biofilm (1), which can be subsequently collected and processed.

As to particular embodiments, a method of producing the inventive culturing system (2) can include providing a circuitous member (4), coupling a biofilm adhesion surface (3) to the circuitous member (4), supporting the circuitous member (4) in a circuitous travel path (8) with an axial element (10), and advancing the circuitous member (4) in the circuitous travel path (8) with a drive mechanism (11).

The circuitous member (4) or the biofilm adhesion surface (3) can be entirely formed of the same material, or alternatively, various portions of the circuitous member (4) or the biofilm adhesion surface (3) can be formed from different materials. The circuitous member (4) or the biofilm adhesion surface (3) can be produced from any of a wide variety of materials, including substantially resiliently flexible materials. By way of non-limiting example, the material can be anti-bacterial, natural, synthetic, textured, substantially smooth, woven, nonwoven, or the like and can include or consist of: cotton, linen, rayon, viscose, ramie, hemp, jute, leather, leather-like material, rubber, rubber-like material, plastic, plastic-like material, acrylic, polyamide, polyester, microfiber, polypropylene, polyvinyl chloride-based materials, silicone-based materials, film, or the like, or combinations thereof.

The circuitous member (4) or the biofilm adhesion surface (3) can be produced from any of a wide variety of processes depending upon the application, such as press molding, injection molding, fabrication, machining, printing, additive printing, or the like, or combinations thereof, as one piece or assembled from a plurality of pieces into an embodiment of the circuitous member (4) or the biofilm adhesion surface (3) or provided as a plurality of pieces for assembly into an embodiment of the circuitous member (4) or the biofilm adhesion surface (3). As to particular embodiments, the circuitous member (4) and the biofilm adhesion surface (3) can be produced as one discrete piece. As to other particular embodiments, the circuitous member (4) and the biofilm adhesion surface (3) can be produced as separate discrete pieces, which can subsequently be coupled together.

As to particular embodiments wherein the circuitous member (4) and the biofilm adhesion surface (3) comprise separate discrete pieces, the biofilm adhesion surface (3) can be coupled to the circuitous member (4) by a variety of methods of joining materials, which can include methods for fixedly joining materials or methods for removably joining materials, including but not limited to, adhering, fastening, cementing, crimping, fusing, gluing, sealing, taping, or the like. Exemplary adhesives can include, as illustrative examples: non-reactive adhesives including drying adhesives, pressure-sensitive adhesives, contact adhesives, and hot adhesives; reactive adhesives including one-part adhesives and multi-part adhesives; natural adhesives; synthetic adhesives; or the like. Exemplary mechanical fasteners can include, as illustrative examples: buckles, buttons, clamps, clips, grommets, hook-and-eye closures, mated hook and loop fasteners, pins, rivets, snap fasteners, staples, stitches, straps, tape, zippers, or the like. As to particular embodiments, the biofilm adhesion surface (3) can be fixedly joined or removably joined to the circuitous member first surface (19), to the circuitous member second surface (20), or to both the circuitous member first surface (19) and the circuitous member second surface (20).

The method of producing the inventive culturing system (2) can further include disposing the circuitous member (4) about a plurality of axial elements (10). As to particular embodiments, the plurality of axial elements (10) can be disposed in fixed spatial relation to dispose the circuitous member (4) in a circuitous member configuration which increases a circuitous member surface area (21) of the circuitous member (4) in fixed volume of space (52) or per unit volume of space (52). As an illustrative example, the circuitous member (4) can be disposed about a plurality of axial elements (10) such that the circuitous member configuration can be defined by a plurality of accordion folds (54), which can accomplished by weaving the circuitous member (4) about the plurality of axial elements (10) such that a portion of the circuitous member (4) can define a plurality of accordion folds (54) and another portion of the circuitous member (4) can be substantially planar and disposed below the portion of the circuitous member (4) defining the plurality of accordion folds (54), proximate the medium biofilm culture medium (7).

The method of producing the inventive culturing system (2) can further include coupling a driven member (58) with the axial element (10), which can be accomplished by a variety of methods of joining materials, which can include methods for fixedly joining materials or methods for removably joining materials, including but not limited to, adhering, fastening, cementing, crimping, fusing, gluing, sealing, taping, or the like. Exemplary adhesives and exemplary mechanical fasteners can include the adhesives and mechanical fasteners previously described.

The method of producing the inventive culturing system (2) can further include providing a biofilm culture medium (7) through which the circuitous member (4) can advance in the circuitous travel path (8). As to particular embodiments, the biofilm culture medium (7) can include an algae culture medium (65).

The method of producing the inventive culturing system (2) can further include providing a receptacle (66) for containing the biofilm culture medium (7), wherein the circuitous member (4) can advance in a circuitous travel path (8) passing through the biofilm culture medium (7) contained within the receptacle (66). The receptacle (66) can be entirely formed of the same material, or alternatively, various portions of the receptacle (66) can be formed from different materials. The receptacle (66) can be produced from any of a wide variety of materials, including, as illustrative example, concrete, ceramic, metal, plastic, glass, fiberglass, or the like, or combinations thereof. The receptacle (66) can be produced from any of a wide variety of processes depending upon the application, such as press molding, injection molding, fabrication, machining, printing, additive printing, or the like, or combinations thereof, as one piece or assembled from a plurality of pieces into an embodiment of the receptacle (66) or provided as a plurality of pieces for assembly into an embodiment of the receptacle (66).

The method of producing the inventive culturing system (2) can further include providing an optical filter (78) disposed to filter light incident upon the biofilm (7) coupled to the circuitous member (4). As to particular embodiments, the optical filter (78) can transmit light having a predetermined wavelength, wherein the wavelength can modulate one or more parameters selected from the group including or consisting of: production of the biofilm (1), rate of growth of the biofilm (1), biomass of the biofilm (1), photosynthesis of the biofilm (1), production of a biochemical component by the biofilm (1), and carbon dioxide (84) consumption by the biofilm (1). As to particular embodiments, the biochemical component can be selected from the group including or consisting of: ethanol, lipids, polyunsaturated fatty acids, omega-3 fatty acids, carbohydrates, proteins, nucleic acids, antioxidants, pigments, carotenoids, beta carotene, lutein, and astaxanthin.

The optical filter (78) can be any of a wide variety of optical filters (78), including conventional optical filters (78) suitable for use in the above context, which will be readily apparent to those of ordinary skill in the art. Additionally, the optical filter (78) can include an optical filter (78) configured to transmit light having a wavelength which can modulate one or more parameters selected from the group including or consisting of: production of the biofilm (1), rate of growth of the biofilm (1), biomass of the biofilm (1), photosynthesis of the biofilm (1), production of a biochemical component by the biofilm (1), and carbon dioxide (84) consumption by the biofilm (1). As to particular embodiments, the biochemical component can be selected from the group including or consisting of: ethanol, lipids, polyunsaturated fatty acids, omega-3 fatty acids, carbohydrates, proteins, nucleic acids, antioxidants, pigments, carotenoids, beta carotene, lutein, and astaxanthin.

The method of producing the inventive culturing system (2) can further include providing a light source (9) which can emit an amount of light incident upon the biofilm (1) coupled to the circuitous member (4), wherein the light source ( ) can be as described above.

The method of producing the inventive culturing system (2) can further include providing a source of carbon dioxide (84), which can be as described above.

The method of producing the inventive culturing system (2) can further include providing a harvesting assembly (95), which can be as described above.

The method of producing the inventive culturing system (2) can further include providing photosynthetic particles (29) which can adhere to the biofilm adhesion surface (3) to form a biofilm (1). As to particular embodiments, the photosynthetic particles (29) can be provided to the biofilm culture medium (7).

As to particular embodiments, the photosynthetic particles (29) can include photosynthetic organisms (30). As to particular embodiments, the photosynthetic organisms (30) can include algae (6), including unicellular algae or multicellular algae. As to particular embodiments, the algae (6) can be selected from the group including or consisting of: Axodine, Bacillariophyceae (for example Phaeodactylum tricornutum), Bryopsidophyceae, Chlorophyceae (for example Haematococcus pluvialis), Cyanophyceae (for example Spirulina), Dinophyceae (for example Crypthecodinium cohnii), Eustigmatophyceae (for example Nannochloropsis or Monodus subterraneus), Labyrinthulea (for example Schizochytrium or Thraustochytrium aggregatum or Ulkenia), Mesostigmatophyceae, Pelagophyceae, Phaeophyceae, Phaeothamniophyceae, Pleurastrophyceae, Prasinophyceae, Raphidophyceae, Synurophyceae, Trebouxiophyceae (for example Chlorella), Ulvophyceae, Xanthophyceae, or the like, or combinations thereof.

As to particular embodiments, a method for culturing a biofilm (1) can include obtaining an inventive culturing system (2) comprising a circuitous member (4), a biofilm adhesion surface (3) coupled to the circuitous member, an axial element (10) which supports the circuitous member (4) in a circuitous travel path (8), and a drive mechanism (11) operable to advance the circuitous member (4) in the circuitous travel path (8); providing a biofilm culturing medium (7) through which the circuitous member (4) can advance in the circuitous travel path (8); providing photosynthetic particles (29) to the inventive culturing system (2), the photosynthetic particles (29) capable of adhering to the biofilm adhesion surface (3) to form the biofilm (1); and exposing the biofilm (1) to a light source (9) filtered by an optical filter (78).

The method for culturing a biofilm (1) can further include harvesting the biofilm (1). As to particular embodiments, the method for culturing a biofilm (1) can further include deriving from the biofilm (1) one or more biochemical components selected from the group including or consisting of: ethanol, lipids, polyunsaturated fatty acids, omega-3 fatty acids, carbohydrates, proteins, nucleic acids, antioxidants, pigments, carotenoids, beta carotene, lutein, astaxanthin, or the like, or combinations thereof. As to particular embodiments, the method for culturing a biofilm (1) can further include deriving biodiesel from the biofilm (1). As to particular embodiments, the method for culturing a biofilm (1) can further include deriving powdered algae from the biofilm (1). The biochemical components, biodiesel, or powdered algae can be derived by conventional methods and systems known to those of ordinary skill in the art.

As to particular embodiments, the method for culturing a biofilm (1) can also be used to culture organisms in a suspension, for example an algae suspension. As an illustrative example, the method for culturing a biofilm (1) can be used to culture a biofilm (1) coupled to the circuitous member (1) advancing through a receptacle (66) containing a biofilm culture medium (7) including an algae suspension.

As can be easily understood from the foregoing, the basic concepts of the present invention may be embodied in a variety of ways. The invention involves numerous and varied embodiments of a culturing system and methods for making and using such culturing systems including the best mode.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “culture” should be understood to encompass disclosure of the act of “culturing”—whether explicitly discussed or not—and, conversely, were there effectively disclosure of the act of “culturing”, such a disclosure should be understood to encompass disclosure of a “culture” and even a “means for culturing.” Such alternative terms for each element or step are to be understood to be explicitly included in the description.

In addition, as to each term used it should be understood that unless its utilization in this application is inconsistent with such interpretation, common dictionary definitions should be understood to included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the culturing systems herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A culturing system comprising:
a circuitous member;
a biofilm adhesion surface coupled to said circuitous member;
an axial element which supports said circuitous member in a circuitous travel path; and
a drive mechanism operable to advance said circuitous member in said circuitous travel path.

2. The culturing system of claim 1, wherein said biofilm adhesion surface couples to a circuitous member first surface.

3. The culturing system of claim 2, wherein said biofilm adhesion surface couples to a circuitous member second surface.

4. The culturing system of claim 3, further comprising a biofilm capable of adhering to said biofilm adhesion surface.

5-13. (canceled)

14. The culturing system of claim 3, wherein said axial element engages a corresponding portion of a circuitous member surface of said circuitous member to support said circuitous member.

15. (canceled)

16. The culturing system of claim 14, further comprising a spacer element disposed between said axial element and said adjacent circuitous member surface.

17. The culturing system of claim 14, said circuitous member disposed about a plurality of said axial elements.

18. The culturing system of claim 17, said plurality of axial elements configured to dispose said circuitous member in a circuitous member configuration which increases a circuitous member surface area of said circuitous member in a fixed volume of space.

19. The culturing system of claim 18, said circuitous member configuration defined by a plurality of accordion folds.

20. The culturing system of claim 19, further comprising a driven member coupled to said axial element.

21. The culturing system of claim 20, wherein said drive mechanism drives said driven member, rotating said driven member about a rotation axis to advance said circuitous member in said circuitous travel path.

22-23. (canceled)

24. The culturing system of claim 21, further comprising a biofilm culture medium through which said circuitous member advances in said circuitous travel path.

25. (canceled)

26. The culturing system of claim 24, further comprising a receptacle containing said biofilm culture medium, said circuitous member advancing in said circuitous travel path passing through said biofilm culture medium contained within said receptacle.

27. The culturing system of claim 24, further comprising an optical filter disposed to filter light incident upon said biofilm coupled to said circuitous member.

28. The culturing system of claim 27, wherein said optical filter transmits light having a predetermined wavelength.

29. The culturing system of claim 28, wherein said wavelength modulates one or more parameters selected from the group consisting of: production of said biofilm, rate of growth of said biofilm, biomass of said biofilm, photosynthesis of said biofilm, production of a biochemical component by said biofilm, and carbon dioxide consumption by said biofilm.

30. The culturing system of claim 29, wherein said biochemical component is selected from the group consisting of: ethanol, lipids, polyunsaturated fatty acids, omega-3 fatty acids, carbohydrates, proteins, nucleic acids, antioxidants, pigments, carotenoids, beta carotene, lutein, and astaxanthin.

31-32. (canceled)

33. The culturing system of claim 29, further comprising a light source which emits an amount of light incident upon said biofilm coupled to said circuitous member.

34-37. (canceled)

38. The culturing system of claim 33, further comprising a source of carbon dioxide.

39-48. (canceled)

49. The culturing system of claim 33, further comprising a harvesting assembly.

50-75. (canceled)
